(19) United States
(12) Patent Application Publication
(10) Pub. No.: US 2021/0085557 A1
(43) Pub. Date: Mar. 25, 2021

(54) DEVICE FOR THE TRANSFORMATION OF A BALLISTIC PRESSURE WAVE INTO A DEFOCALISED ACOUSTIC SHOCK WAVE

(71) Applicants: Daniele FORONI, Napoli (IT); Marco MINUCCI, Napoli (IT); Luca PALMA, Napoli (IT)

(72) Inventors: Daniele FORONI, Napoli (IT); Marco MINUCCI, Napoli (IT); Luca PALMA, Napoli (IT)

(21) Appl. No.: 16/956,603

(22) PCT Filed: Jun. 12, 2018
(86) PCT No.: PCT/IB2018/054219

§ 371 Date: Jun. 21, 2020

(30) Foreign Application Priority Data
Dec. 22, 2017 (IT) 102017000149512

(51) Int. Cl. A61H 23/00; A61H 23/02

(52) U.S. Cl. CPC A61H 23/008; A61H 2201/1654; A61H 2201/0153; A61H 23/0245

(57) ABSTRACT

A device (5) it is provided for cooperating with a handpiece (1) of a ballistic waves generating system for the treatment of a portion of tissue (4) of a patient, the handpiece (5) comprising a supporting member (30) on top of which an end member (3) it is arranged to come into contact with the said portion of tissue (4) of the patient, the device (5) it is characterized in that it comprises supporting means (50) of terminal means (51), said supporting means (50) being adapted to be mounted onto said supporting member (30), and said terminal means (51) being able to come into contact with the tissue portion (4) of the patient.

### DESCRIPTION

The present invention relates to a device for transforming a ballistic pressure wave and, more precisely, to a device comprising a terminal of a ballistic wave generating handpiece for the transformating of a ballistic pressure wave in a devocalised acoustic shock wave.

### STATE OF THE ART

As it is already known, shock waves are acoustic waves that accompany our daily life without being noticed, i.e. the noise caused by thunders or an explosion are examples wherein shock waves play an important role.

Thanks to the mechanical feature of shock waves, energy can be transmitted over long distances. For example, an airplane flying in the sky generates a loud noise which may clink glasses in a cupboard in an apartment. Thus, the shock wave generated by the airplane transmits energy into the air up to the glass.

On the other hand, shock waves have been used in different medical fields for about 20 years. For example, in the urological field (lithotrity of kidney stones), in the rehabilitative orthopaedic field, in the cardiological field and in the aesthetic field.

On the other hand, according to the type of wave generator, shock waves can be divided into 3 types:

1) Focused shock waves

This type of shock waves has a high energy peak, rapid pressure rise and short duration. This type of shockwave gives the possibility to focus the pulse at an intracorporal level at an adjustable distance.

2) Defocalised shock waves

This type of shock waves has a lower peak of energy and less focalization of the impulse.

3) Radial or ballistic shock waves

This type of shock waves has a low energy peak and it is impossible to focus the impulse.

Furthermore, in addition to the difference between two main types of equipment used for the generating of focused or radial waves, it is necessary to highlight here some technical aspects related to the production of this type of wave, as follows:

a) Focused shock waves

They are produced by three different types of generators:
piezoelectric generator;
electromagnetic generator;
electro-hydraulic generator;

with consequent methods of producing acoustic energy.

The focused shockwaves, as such, in addition to converging the energy in a specific target, modify the dimensions of the therapeutic focus according to the energetic level used.

b) Radial shock waves
Radial shockwaves do not focus the impulse and therefore cannot converge energy in depth;
Radial shock waves do not allow the use of high power energy, as opposed to said focused shock waves;
The use of radial shock waves it is mainly limited to superficial muscle-tendon disorders.

c) Ballistic shock waves

The use of ballistic shock waves it is a method that uses a special handpiece at the end of which there is provided a terminal body which transmits the pressure effect transferring it into the skin and the first underlying layer of tissue. For example, there are some gun-shaped models wherein a fired bullet hits the terminal body generating thereby the pressure wave onto the patient skin.

At the moment of impacting the patient body, the energy it is transferred to the contacting point and from there it is diverged in a radial wave shape, reaching an average depth of about 20-22 mm.

With particular reference to the use of this type of systems using ballistic waves, it is necessary to point out here that they have some limitations.

A first disadvantage lies in the fact that current ballistic systems cannot be used in specific sensitive and delicate areas of the human body such as, for example, neck, cheekbones, lips, skull, genital organs, décolleté and proximity of large blood vessels.

Another disadvantage of the current ballistic systems lies in the fact that it is impossible to treat pain sensitive areas or where the bone tissue resonates.

The object of the present invention it is to solve the aforementioned disadvantages by providing a device for the use on a terminal part of a ballistic wave generating handpiece system, for the treating of a patient tissue through which the pressure components are eliminated, and by which device the pressure components are eliminated during the transmission of pressure and acoustic energy (shock waves), allowing only the acoustic component to pass through the patient's tissue.

### BRIEF DESCRIPTION OF THE INVENTION

The present invention provides a device for being deployed onto a terminal of an handpiece of a ballistic wave generation system for the treatment of a tissue area of a patient, the handpiece comprising a supporting member on top of which a terminal member it is arranged and suitable for contacting the skin/tissue portion of the patient, the device being characterized in that it comprises supporting means for the supporting of terminal means, said supporting means being adapted to be mounted onto said supporting member, and said terminal means being adapted to contacting said patient tissue portion.

Thus, the present invention provides a device for being used on a terminal part of an handpiece of a ballistic wave generating system for the treating of a patient tissue substantially according to the appended claims.

### DETAILED DESCRIPTION OF THE INVENTION

A detailed description of a preferred embodiment of the device of the present invention will now be given, by way of a non-limiting example, and with reference to the accompanying figures, wherein:

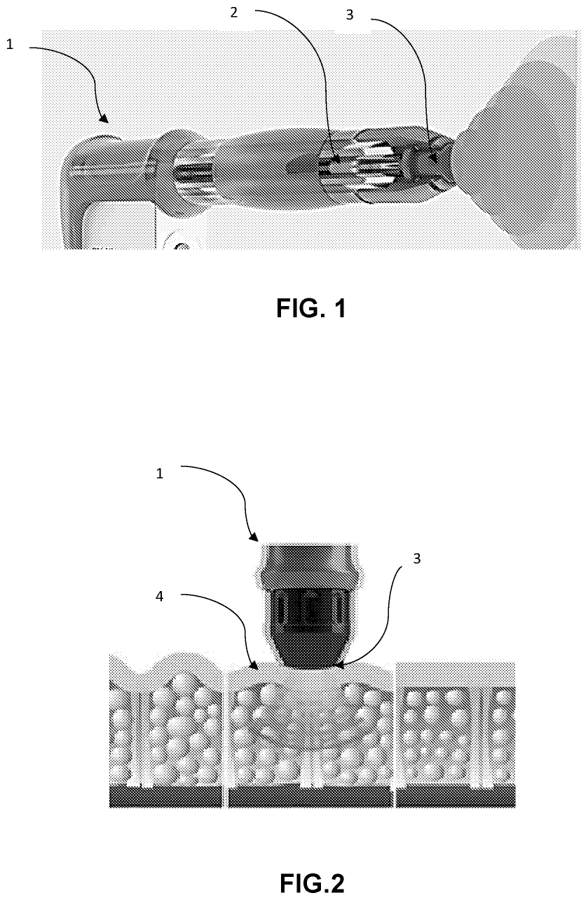

FIG. 1 it is a schematic perspective view illustrating the operating principle of functioning of a generic compressed air handpiece for generating a pressure wave according to the state of the art;

FIG. 2 it is a schematic view illustrating how energy it is transferred from the handpiece onto a patient's tissue with a ballistic shock wave generating system of the state of the art;

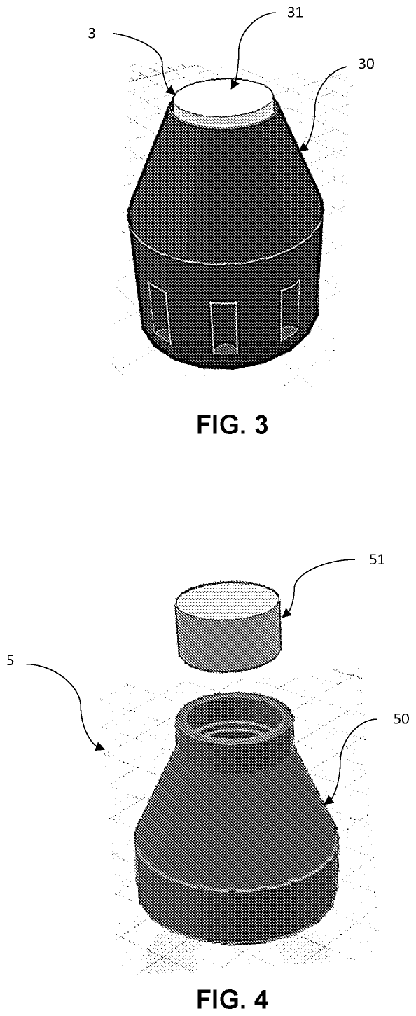

FIG. 3 it is a perspective view showing a generic compressed air terminal part of an handpiece for the generating of a pressure wave according to the state of the art;

FIG. 4 it is an exploded view of the device of the present invention; and

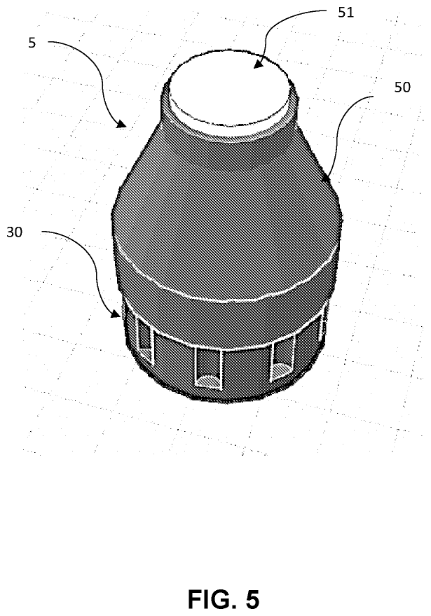

FIG. 5 it is a perspective view of the device of the present invention mounted onto a generic handpiece in a non-functional condition.

Referring now to FIG. 1, a generic handpiece 1 for the generation of a ballistic shock waves according to the state of art there is shown.

According to this embodiment, the handpiece 1 it is pistol shaped wherein a bullet 2 it is fired with compressed air and hits a terminal member 3, thus generating the pressure wave which propagates in a radial direction.

Referring to FIG. 2, there is schematically shown in detail the propagation of a pressure wave into a portion of tissue 4 of a patient body through the handpiece 1 of FIG. 1.

As it may be noted in the figure, the terminal member 3 resting onto the portion of tissue 4 to be treated transfers the energy from the contacting point towards the inside of the tissue 4, causing said energy to diverge in a radial shaped wave (typically reaching an average depth of about 20-22 mm).

Referring now to FIG. 3, there is illustrated therein in detail the terminal part of the handpiece 1 of FIG. 1 of the state of the art.

As can be seen in the figure, the handpiece comprises a frustoconical shaped supporting member 30 on the top of which the terminal member 3 it is arranged in such a way that the same may come into contact with the patient's tissue portion (the latter not shown).

The arrangement it is such that the energy coming from the shock wave generated by the bullet 2 (not in the figure) when hits the rear part of the terminal member 3 it is transferred through the surface 31 of the terminal member 3 contacting the tissue portion of the patient (not shown). The frusto-conical supporting member it is configured to be mounted onto the pistol end part of the handpiece 1 (the latter being not shown in the figure).

Referring now to FIG. 4, there is shown therein a device according to the present invention in an exploded view.

According to the present invention, there is provided a device 5 which comprises a supporting member 50 having a frustoconical shape and a cylindrical terminal member 51 arranged on the top of said frustoconical supporting member 50. The terminal member 51 it is configured to come into contact with the skin of the patient.

According to the invention, the terminal member 51 it is constituted by a substantially cylindrical body and made of flexible material and containing inside thereof a gelatinous or liquid substance, and in any case a fluid matter.

The terminal member 51 thus formed it is mounted onto the end part of the supporting member 50.

Referring now to FIG. 5, there is shown the device 5 of the present invention mounted onto the terminal member 3 of a generic handpiece (last not in the figure).

According to the invention, the supporting member 50 has a frustoconical shape suitable to cooperate with the supporting member 30 of the handpiece 1. The shape of the supporting member 50 of the device of the present invention it is complementary with the shape of the supporting member 30 of the handpiece. The supporting member 50 of the device 5 can be inserted (coupled) onto the terminal member 30 of the handpiece 1. In this condition, the terminal member 51 of the device 5 of the present invention it is in contact with the surface 31 of the terminal member 3 of the handpiece and therefore the latter can transfer the energy of the shock wave to the former.

More precisely, during operation the terminal member 3 of the handpiece 1 is in contact with the terminal member 51 of the device 5 of the present invention, while the terminal member 51 it is in contact with the patient's skin.

According to this configuration, the energy travels through the handpiece 1 through the two terminal members 3 and 51 (i.e., terminal members of the handpiece 1 and the device 5 of the invention) thereby to be transmitted through the patient's skin.

It will be apparent to those skilled in the art that the present configuration, i.e. by interposing a terminal member 51 made of fluid material between the terminal member 3 of the handpiece 1 and the skin 4 of the patient, allows the pressure component to be filtered/dampened leaving only the acoustic component of the shock wave passing through.

Furthermore, it will be apparent to those skilled in the art that according to an alternative embodiment of the device 5 of the present invention, it is possible to make the operative arrangement above described also with a single piece supporting member 50 which comprises integrally a first and a second terminal member 3 and 51 abutted onto a single supporting member 50 mounted onto the handpiece 1.

The device 5 of the present invention has several advantages.

According to a first advantageous aspect of the present invention when the device 5 it is combined with a ballistic system it gives the possibility of having in a single machine the feature of generating both defocalised pressure and acoustic waves, and also in a separated manner.

According to another advantageous aspect of the present invention, the acoustic waves generated with the present device 5 have a depth of penetration in the tissues which is lower than the traditional systems. This feature makes it possible to overcome the application limits of the handpieces of the state of the art employing acoustic and pressure waves.

According to a further advantageous aspect of the present invention, with the present device 5 it is possible to fill the application gap of the handpieces of the state of the art, given the low penetration of the waves in the tissue the device of the present invention it is particularly suitable for applications in formerly untreatable body areas, such as for example: neck, cheekbones, lips, skull, genital organs, décolleté, and near large blood vessels.

The present invention finds application in all the fields where patient tissue treatment is required for a deep of no more than 15 mm.

Examples

In the rehabilitation field:

Especially painful superficial tendinitis (epicondylitis, epitrocleitis, hand tendinitis, foot, Dupuytren's disease, De Quervain's disease, carpal tunnel, snap finger, etc.)

In the aesthetic field:

face, lips, forehead, cheekbones, neck, scalp, décolleté, abdomen, buttocks, thighs, arms

Pathologies of the female genital apparatus: rejuvenation Vulnerary diseases

Pathologies of the male genital apparatus:

Erectile dysfunction, induratio penis plastic.

In the dermatological field:

bedsores and aseptic ulcers

1. Device (5) for cooperating with an handpiece (1) of a ballistic wave generation system for the treatment of a tissue portion (4) of a patient, the handpiece (1) comprising a supporting member (30) on the end part of which a terminal member (3) it is arranged to come into contact with the said portion of tissue (4) of the patient,
characterized in that it further comprises supporting means (50) for supporting terminal means (51), said supporting means (50) being adapted to be mounted onto said supporting member (30), and said terminal means (51) being suitable for come into contact with said tissue portion (4) of the patient.

2. Device (5) according to the preceding claim, wherein said supporting means comprises a supporting member (50) having a shape which it is complementary to the shape of said supporting member (30) of said handpiece (1).

3. Device (5) according to claim 2, wherein said terminal means comprises a terminal member (51), said terminal member (51) being mountable on the top of said supporting member (50).

4. Device (5) according to the preceding claim, wherein said terminal member (51) consists of a body made of flexible material and containing a fluid matter.

5. Device (5) according to any of the preceding claims, wherein when said device (5) it is mounted on said handpiece (1) the terminal member (51) of the device (5) it is in contact with the terminal member (3) of the handpiece (1).

6. Device (5) according to any of the preceding claims, wherein said device (5) comprises a supporting member (50) having a frustoconical shape and a cylindrical terminal member (51) arranged on the top of said frustoconical shaped supporting member (50).

7. Device (5) according to the preceding claim, wherein said terminal member (51) of said device (5) it is constituted by a substantially cylindrical shaped body made of flexible material and containing a fluid matter.

8. Device (5) for cooperating with an handpiece (1) of a ballistic wave generation system for the treatment of a tissue portion (4) of a patient, the handpiece (1) comprising a supporting member (50) containing a terminal member (3),
characterized in that it further comprises terminal means (51) mounted on the top of said supporting member (50) and arranged for cooperating with said terminal member (3) and said terminal means (51) being suitable for coming into contact with said tissue portion (4) of the patient, the arrangement being such that the energy coming from the handpiece (1) it is transferred to said tissue portion (4) of a patient through said end member (3) and said terminal means (51).

9. Device (5) according to the preceding claim, wherein said terminal means comprise a second terminal member (51), said second terminal member (51) being mountable on the top of said supporting member (50).

10. Device (5) according to the preceding claim, wherein said second terminal member (51) consists of a body made of flexible material and containing a fluid matter.
